(19) United States
(12) Patent Application Publication
(10) Pub. No.: US 2026/0232392 A1
(43) Pub. Date: Aug. 13, 2026

(54) TORQUE SENSORS FOR ROBOTIC SURGICAL SYSTEMS

(71) Applicant: Covidien LP, Mansfield, MA (US)

(72) Inventors: Ranjan Kumar Mishra, Orange, CT (US); Michael A. Zemlok, Prospect, CT (US); Eric J. Taylor, Southington, CT (US)

(21) Appl. No.: 19/155,725

(22) PCT Filed: Feb. 9, 2024
(86) PCT No.: PCT/IB2024/051236

§ 371 Date: Aug. 12, 2025

Related U.S. Application Data
(60) Provisional application No. 63/446,393, filed on Feb. 17, 2023.

(51) Int. Cl. A61B 34/30; A61B 17/00; A61B 90/00

(52) U.S. Cl. CPC A61B 34/30; A61B 17/00; A61B 90/06; A61B 2017/00398; A61B 2017/00477; A61B 2034/305; A61B 2090/066

(57) ABSTRACT

A surgical system includes an instrument drive assembly supporting a motor assembly and a torque sensor. The torque sensor is coupled to the motor assembly and includes an output coupler, an input coupler, and a shaft assembly that connects the output coupler to the input coupler. The output coupler is configured to engage with a surgical instrument. The input coupler is engaged with the motor assembly. The shaft assembly includes an outer shaft mounted on an inner shaft. The inner shaft is movable relative to the outer shaft to impart a first torque, and the inner shaft is movable with the outer shaft to impart a second torque.

### CROSS-REFERENCE TO RELATED APPLICATION

This application claims the benefit of U.S. Provisional Patent Application Ser. No. 63/446,393, filed Feb. 17, 2023, the entire content of which is incorporated herein by reference.

### BACKGROUND

Robotic surgical systems have been used in minimally invasive medical procedures. During such a medical procedure, the robotic surgical system is controlled by a surgeon interfacing with a user interface. The user interface allows the surgeon to manipulate an end effector that acts on a patient. The user interface includes an input controller or handle that is moveable by the surgeon to control the robotic surgical system.

The end effectors of the robotic surgical system are positioned at the end of robotic arms. Each end effector is manipulated by a control drive unit that supports a motor assembly that is operable to move the end effector about a respective axis or to perform a particular function of the end effector (e.g., approximate, pivot, etc. jaws of the end effector). The motor assembly can include a plurality of drive motors with each drive motor being associated with a respective degree of freedom or function of the end effector. The drive motors can be coupled to torque sensors to measure force applied by the drive motors.

### SUMMARY

In accordance with an aspect of the present disclosure, a robotic surgical system includes an instrument drive assembly supporting a motor assembly, a surgical instrument operably coupled to the instrument drive assembly, and a torque sensor. The torque sensor is supported between the motor assembly and the surgical instrument. The torque sensor includes an output coupler, an input coupler, and a shaft assembly that connects the output coupler to the input coupler. The shaft assembly includes an outer shaft mounted on an inner shaft. The inner shaft is movable relative to the outer shaft to impart a first torque, and is movable with the outer shaft to impart a second torque.

In aspects, the first torque may be lower than the second torque. The inner shaft may include an output arm and an input arm that are separated by a connecting arm. The connecting arm may have a smaller diameter than the output arm and the input arm. The inner shaft may include a first spline supported on the output arm and a second spline supported on the input arm. The outer shaft may define a first spline slot within which the first spline seats and a second spline slot within which the second spline seats. The first spline and the first spline slot may be torsionally locked together. The second spline may be configured to slide through the second spline slot as the inner shaft rotates relative to the outer shaft. When the second spline slides through the second spline slot, the inner shaft may be positioned for angular displacement relative to the outer shaft. The second spline may be engageable with a sidewall of the outer shaft that defines the second spline slot. When the second spline engages the sidewall, the outer shaft and the inner shaft may rotate together.

In aspects, the robotic surgical system may further include at least one rotational angle sensor coupled to the shaft assembly.

According to another aspect, this disclosure is directed to a surgical system. The surgical system includes an instrument drive assembly supporting a motor assembly, and a torque sensor coupled to the motor assembly. The torque sensor includes an output coupler configured to engage with a surgical instrument, an input coupler engaged with the motor assembly, and a shaft assembly that connects the output coupler to the input coupler. The shaft assembly includes an outer shaft mounted on an inner shaft. The inner shaft is movable relative to the outer shaft to impart a first torque and is movable with the outer shaft to impart a second torque.

According to still another aspect, this disclosure is directed to a torque sensor for a robotic surgical system. The torque sensor includes an output coupler configured to engage with a surgical instrument, a first rotational angle sensor coupled to the output coupler, an input coupler configured to engage with a motor assembly, a second rotational angle sensor coupled to the input coupler, and a shaft assembly defining a longitudinal axis and connecting the output coupler to the input coupler. The shaft assembly includes an outer shaft mounted on an inner shaft. The inner shaft is movable relative to the outer shaft through a first angular displacement, and is movable with the outer shaft through a second angular displacement that is greater than the first angular displacement. The first and second rotational angle sensors are configured to cooperate with one another to enable the robotic surgical system to determine torque imparted on the robotic surgical system as the shaft assembly rotates about the longitudinal axis.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate aspects of this disclosure and, together with a general description of this disclosure given above, and the detailed description given below, explain the principles of this disclosure, wherein:

FIG. 1 is a perspective view of a robotic surgical system being used for a surgical procedure on a patient in accordance with the principles of this disclosure;

FIG. 2 is an enlarged, perspective view of a torque sensor of the robotic surgical system of FIG. 1 from an output end of the torque sensor;

FIG. 3 is a perspective view of the torque sensor of FIG. 2 from an input end of the torque sensor;

FIG. 4 is a perspective view, with parts separated, of the torque sensor as arranged in FIG. 2;

FIG. 5 is an enlarged, perspective view of the indicated area of detail shown in FIG. 4:

FIG. 6 is a perspective view of an outer shaft of the torque sensor;

FIG. 7 is an enlarged, cross-sectional view of the torque sensor as taken along section line 7-7 shown in FIG. 2;

FIG. 8 is an enlarged, cross-sectional view of the torque sensor as taken along section line 8-8 shown in FIG. 7;

FIGS. 9 and 10 are enlarged, progressive, cross-sectional views of the torque sensor as taken along respective section lines 9-9 and 10-10;

FIG. 11 is a longitudinal cross-sectional view of a drive assembly of the robotic surgical system of FIG. 1, the view showing a servo-drive motor coupled to another torque sensor in accordance with the principles of this disclosure; and

FIG. 12 is a cross-sectional view of FIG. 11 as taken along section line 12-12.

### DETAILED DESCRIPTION

Aspects of this disclosure are described in detail with reference to the drawings, in which like reference numerals designate identical or corresponding elements in each of the several views. As used herein, the term “distal” refers to that portion of structure closer to a patient, while the term “proximal” refers to that portion of structure, farther from the patient. As used herein, the term “clinician” refers to a doctor, nurse, or other care provider and may include support personnel and/or equipment operators. As used herein in connection with torque ranges, the term “about” is indicative of tolerance limits defined by plus or minus ten percent of each end point of the referenced torque range.

In the following description, well-known functions or constructions are not described in detail to avoid obscuring the present disclosure in unnecessary detail.

Robotic surgical systems have been used in minimally invasive medical procedures. Such procedures may be referred to as what is commonly referred to as “Telesurgery.” These robotic surgical systems have one or more surgical instruments removably coupled thereto. Such surgical instruments include, for example, endoscopes, electrosurgical forceps, cutting instruments, staplers, graspers, electrocautery devices, or any other endoscopic or open surgical devices. Prior to or during use of the robotic surgical system, various surgical instruments can be selected and connected to the robotic surgical system for selectively operating end effectors of the connected surgical instruments.

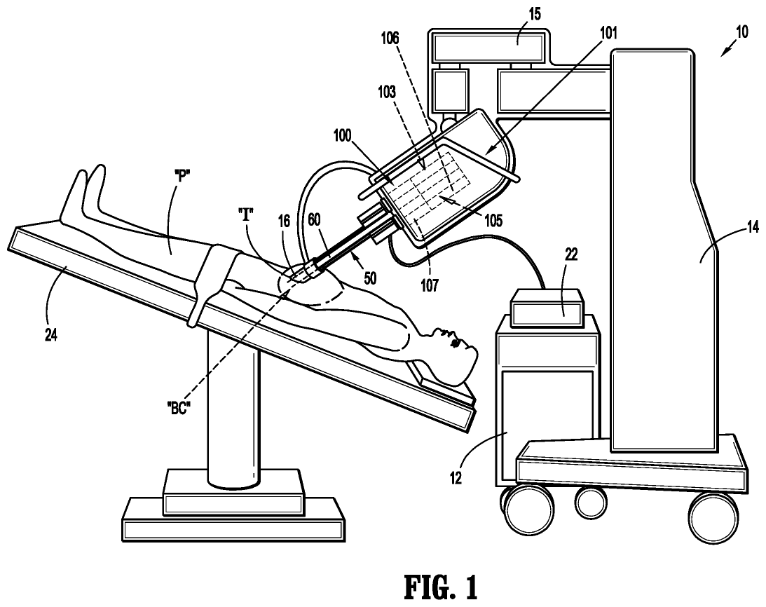

With reference to FIG. 1, a robotic surgical system is shown generally at 10. Robotic surgical system 10 employs various robotic elements to assist the clinician and allow remote operation (or partial remote operation) of surgical instruments 60 of surgical instrument systems 50 of robotic surgical system 10. Various controllers, circuitry, robotic arms, gears, cams, pulleys, electric and mechanical motors, etc. may be employed for this purpose and may be designed with surgical system 10 to assist the clinician during an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

Robotic surgical system 10 includes a workstation 12 and an instrument cart 14. Instrument cart 14 supports a control drive assembly 100 on a setup arm assembly 15 that is selectively movable relative to instrument cart 14. Control drive assembly 100 includes one or more surgical instrument systems 50 mounted on a control drive unit 101 supported on setup arm assembly 15. Control drive unit 101 is movable relative to cart 14 and houses an instrument drive assembly 103 for manipulating surgical instrument systems 50 and/or independent surgical instruments 60 thereof with the assistance of, for example one or more computing devices or controllers.

Surgical instrument system 50 further includes a surgical portal assembly 16 configured to receive, for instance, surgical instruments 60 for accessing a body cavity “BC” of a patient “P.” In particular, surgical portal assembly 16 can be inserted through an incision “I” and into the body cavity “BC” of the patient “P”.

Workstation 12 includes an input device 22 in communication with control drive unit 101 for use by a clinician to control surgical portal assembly 16 and surgical instrument systems 50 (and surgical instruments 60 of instrument systems 50) via an instrument drive assembly 103 for performing surgical operations on the patient “P” while the patient “P” is supported on a surgical table 24, for example. Input device 22 is configured to receive input from the clinician and produces input signals. Input device 22 may also be configured to generate feedback to the clinician. The feedback can be visual, auditory, haptic, or the like. Instrument drive assembly 103 includes a motor assembly 105 having motors 106 and torque sensors 107 coupled to motors 106 for determining torque delivered to motors 106. Torque sensors 107 may be in the form of an in-line rotating shaft torque sensor configured to eliminate noise by isolating the cantilever effect of drive actuators of motor assembly 105.

The torque sensors or transducers of this disclosure are configured to precisely and accurately measure force applied by drive motors. The torque sensors can measure torque when the drive motors are active and inactive such that the torque sensors can measure pretension in the drive cables and can be used to limit forces experienced by the drive cables. It is contemplated that the torque sensors can be used to provide increased fidelity or precision in control of drive motors for surgical devices and feedback in the control of drive motors for the surgical devices including, but not limited to, staplers, wristed or cable-controlled devices, energy-based devices, harmonic devices, rod-actuated devices, graspers, knives, scissors, dissectors, drills, saws (linear or orbital), tacker, hernia anchor and clip devices, and biopsy devices. It is also contemplated that the torque sensors can be part of a sterile interface module (SIM) for driving endoscope rotation, endoscope manipulation, linear drive mechanisms, screw drive mechanism, capstan driven cable tension mechanisms, linear driven cable tension mechanisms, gear driven mechanism, and belt driven mechanisms or the like. It will be appreciated that a SIM maintains a sterile interface while enabling transmission of rotational and/or translational forces and transmission of electrical signals (e.g., power, control, feedback, etc.) between driving mechanisms and driven mechanisms.

The increased fidelity or precision control and feedback may be advantageous for controlling functions of surgical devices including, but not limited to: limiting articulation or position load limits induced or driven by the system; limiting direction overload induced or driven by the system; limiting clamping pressures, load, or direction induced or driven by the system; limiting wristed movements or direction load induced or driven by the system; rotating a device, a device shaft, or an end effector including driving belt driven rotation gear motors; retracting loads to confirm proper function by consistent loading or back drive; recognizing end stops and distal limit positions; limiting drive actuation or load thresholds; firing implantable fasteners including staples, tacks, or clips by verifying formation and formation quality; determining dissection or jaw spreading/opening loads; determining collisions through mechanical shock or heavy load biased or thresholds; determining absence of a reload or an implantable (staple, tack, or clip); activating a knife or cutting mechanism back drive; locking out a device prevent firing in the absence of an implantable; determining stapler length through a force slope increase that corresponds to the device reload length or stroke; back driving torque from a SIM back out device activation that creates additional drag on one or more target drives; setting staple or cutting load limits to prevent damage or fining over undesired tissue, bone, ligament, tubes staple lines, or other devices or implantable fasteners; activating harmonic devices; monitoring for undesired vibrations or inconsistent or irregular back drive loads; and calibrating cannulas with instruments and devices. The increased fidelity or precision control and feedback can be used in a variety of configurations to perform a variety of functions including, but not limited to: confirming that a drive is properly coupled by axial loads in a spring loaded SIM coupler; identifying a device or approach of a distal limit for a mechanical feature or bump; monitor the degradation of drive efficiency to control or limit device life; limiting load spikes to extend device life; providing additional fidelity for haptic reaction forces or vibratory feedback of end user controls; preventing undesired loads on a device or end effector; limiting tension in belts and cables to prolong fatigue life; initializing, homing, calibrating, testing, or confirming the type of device for a motor drive current feedback, rotational encoder, linear encoder, linear load sensor, linear switch, or position sensor; monitoring vibration or backlash to determine drive coupler wear and degradation; manage end of life by monitoring backlash range, belt tension back drive, or belt tension vibrations; monitoring back drive loads induced on the end effector or reload of a device; and monitoring end effector loads back driven by the end user or through collisions on applied loads with the patient or other devices to allow feedback to the end user or to stop the system if a load limit is approached or surpassed.

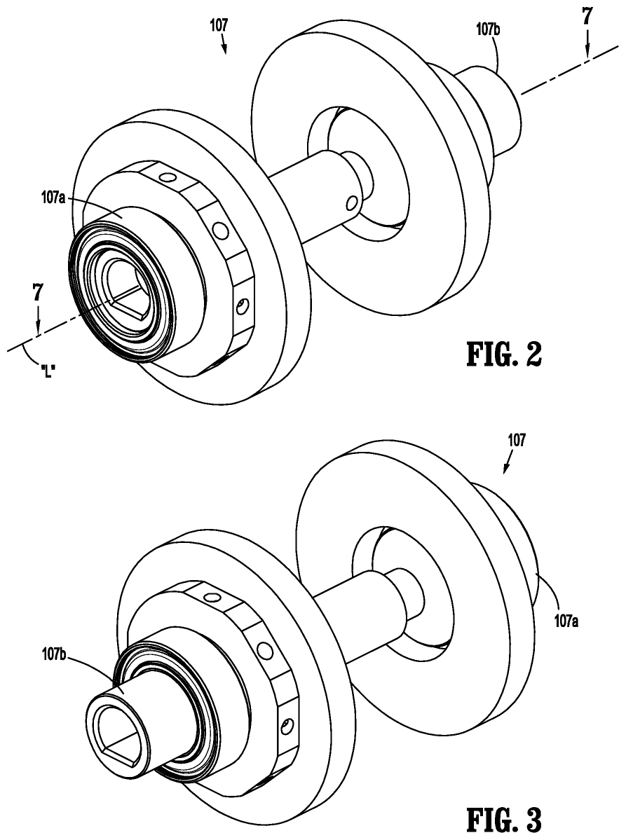

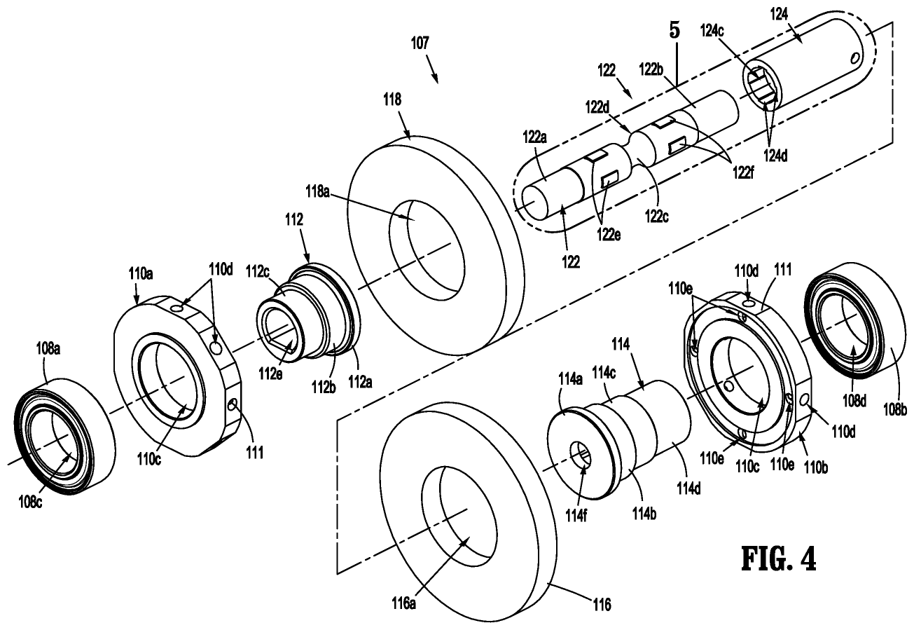

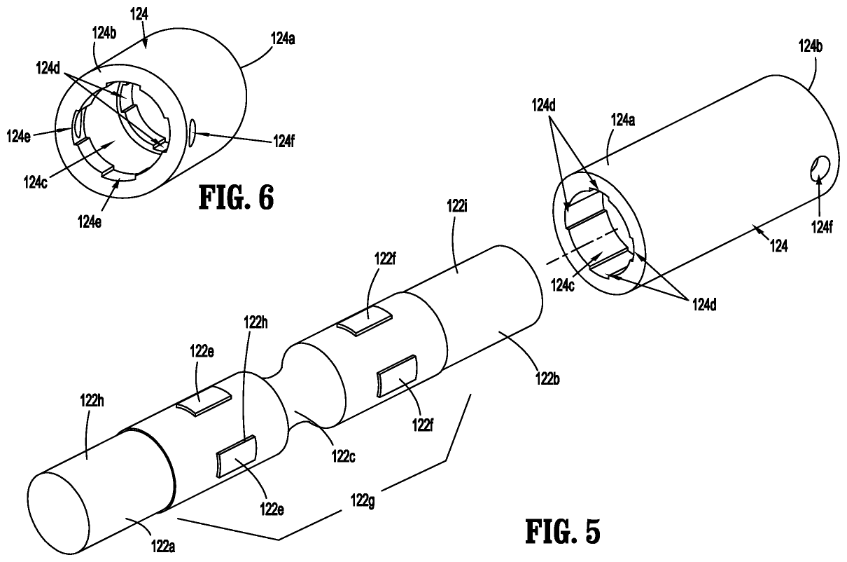

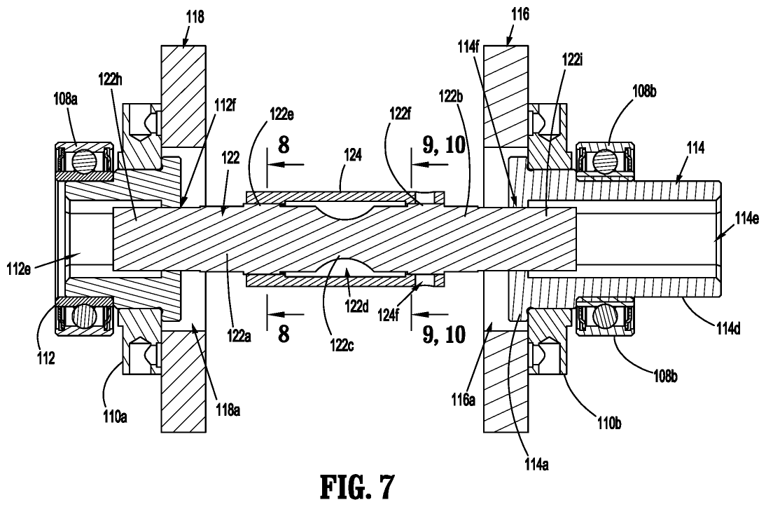

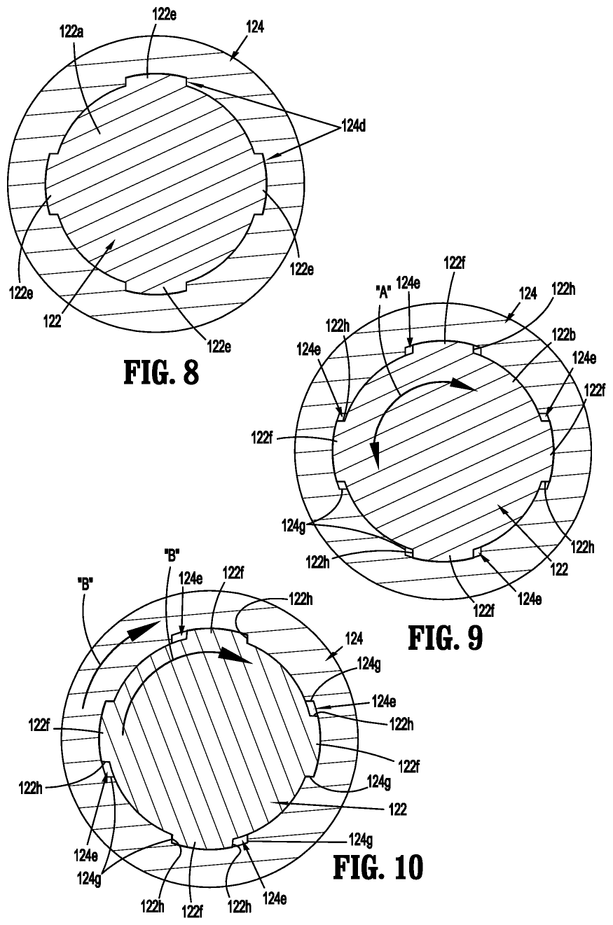

With reference now to FIGS. 2-10, each torque sensor 107 defines a longitudinal axis “L” and includes an output end portion 107a for coupling to a driven assembly (not explicitly shown) of surgical instrument 60 that operates surgical instrument 60, and an input end portion 107b for coupling to one of motors 106 of instrument drive assembly 103 for imparting rotational drive force from motor 106 through torque sensor 107 to output end portion 107a of torque sensor 107. Torque sensors 107 are configured to provide fidelity or precision control and feedback regarding drive forces from instrument drive assembly 103 to surgical instrument 60. Each torque sensor 107 includes an output ball bearing 108a, and input ball bearing 108b, an output mounting member 110a, an input mounting member 110b, an output coupler 112, an input coupler 114, a primary rotational angle sensor 116, a secondary rotational angle sensor 118, which may be identical to primary rotational angle sensor 116, and a shaft assembly 120. Primary and secondary rotational angle sensors 116, 118 are disposed in electrical communication with the one or more controllers (not explicitly shown) of robotic surgical system 10 for providing angular positioning data from torque sensor 107, resulting from rotation of shaft assembly 120, to such controllers.

Output ball bearing 108a of torque sensor 107 defines a central opening 108c for receiving output coupler 112 therethrough. Output ball bearing 108a is configured to facilitate rotation of output coupler 112 about longitudinal axis “L” as shaft assembly 120 rotates about longitudinal axis “L”. Similarly, input ball bearing 108b of torque sensor 107 defines a central opening 108d for receiving input coupler 114 therethrough. Like output ball bearing 108a, input ball bearing 108b is configured to facilitate rotation of input coupler 114 about longitudinal axis “L” to cause shaft assembly 120 to rotate about longitudinal axis “L” when input coupler 114 is coupled to motor assembly 105.

Output mounting member 110a defines a central passage 110c therethrough and a plurality of outer apertures 110d at spaced-apart locations along respective outer surfaces of output and input mounting members 110a, 110b. Output mounting member 110a further defines a plurality of inner apertures 110e at spaced-apart locations aligned with, but in transverse relationship with, outer apertures 110d. The plurality of inner apertures 110e are disposed in communication with the plurality of outer apertures 110d and each is configured to receive fasteners or fastener assemblies 111 for securing output mounting member 110a and input mounting member 110b to respective primary and secondary rotational angle sensors 116, 118. In particular, angle sensors 116 and 118 are axially and radially secured through mounting members 110b and 110a, respectively, to input and output ends of rotating shaft assembly 120. Input mounting member 110b includes the same structure as output mounting member 110a and is disposed in mirrored relationship with output mounting member 110a on the opposite side of each respective torque sensor 107.

Output coupler 112 of torque sensor 107 includes: a flange 112a disposed at an input end of output coupler 112 that is receivable within a central passage 118a of secondary rotational angle sensor 118; a first segment 112b extending from flange 112a toward an output end of output coupler 112 for supporting output mounting member 110a; and a second segment 112c extending from first segment 112b toward the output end of output coupler 112 and having a smaller diameter than first segment 112b for supporting output ball bearing 108a. First and second segments 112b, 112c have smaller diameters than flange 112a. Outer coupler 112 defines a non-circular passage 112e (e.g., D-shaped) on the output end of outer coupler 112 for receiving a driven member (not shown) of surgical instrument 60 and for imparting rotation on the driven member when coupled to the driven member. Outer coupler 112 further defines a shaft opening 112f on the input end of output coupler 112 for receiving an outer coupling end 122h of shaft assembly 120. Shaft opening 112f may be circular and/or non-circular but is configured to fixedly (e.g., non-rotatably) couple to outer coupling end 122h of shaft assembly 120.

Input coupler 114 of torque sensor 107 includes: a flange 114a on an output end of input coupler 114 that is receivable within a central passage 116a of primary rotational angle sensor 116; a first segment 114b extending from flange 114a toward an input end of input coupler 114 for supporting input mounting member 110b; a second segment 114c extending from first segment 114b toward the input end of input coupler 114 and having a smaller diameter than first segment 114b for supporting input ball bearing 108b; and a third segment 114d extending from second segment 114c toward the input end of input coupler 114 and having a smaller diameter than second segment 114c. Input coupler 114 further defines a drive opening 114e (e.g., non-circular) on the input end of input coupler 114 for receiving a drive or output end of motor 106, and a shaft opening 114f on the output end of input coupler 114 for receiving an inner coupling end 122i of shaft assembly 120. Shaft opening 114f may be circular and/or non-circular but is configured to fixedly (e.g., non-rotatably) couple to inner coupling end 122i of shaft assembly 120.

Shaft assembly 120 of torque sensor 107 includes an inner shaft 122 and an outer shaft 124 that can be integrated or assembled onto inner shaft 122. Outer shaft 124 can be a hollow shaft or sleeve or tube or any external form desired that enables the inner form that is mated to inner shaft 122 or sensor structure to produce shaft assembly 120. Shaft assembly 120 provides dual flexure and is configured to provide a wide range of torque sensing with a large fatigue torque limit. Advantageously, such dual flexure enables shaft assembly 120 be shorter than traditional torque sensors because it eliminates the need of combining torque sensing range and fatigue torque. In aspects, inner shaft 122 can be a one-piece machined component or may be a multi-component device joined by any combination of keyed features, a press-fit single flat surface or multiple splines, welding, machine threading, pinning, or fastening (e.g., an array of fasteners). Inner shaft 122 may be machined or manufactured through additive manufacturing. Inner shaft 122 includes an output arm 122a and an input arm 122b that are coupled together by a connecting arm 122c having a smaller diameter than output arm 122a and input arm 122b to enable dual flexure. In particular, connecting arm 122c enables input arm 122b to rotate relative to output arm 122a when low torque flexure is applied to inner shaft 122. Connecting arm 122c is configured to facilitate low torque flexure along a central portion thereof where a diameter of connecting arm 122c is smallest, and configured to facilitate high torque flexure at an output end thereof where the diameter of connecting arm 122c is largest and connecting arm 122c transitions to output arm 122a. Inner shaft 122 further defines an annular channel 122d about a circumference of connecting arm 122c. Output arm 122a further includes a plurality of first splines 122e disposed at spaced-apart locations about a circumference of output arm 122a and which extend longitudinally along inner shaft 122 for torsionally locking output arm 122a to outer shaft 124. Input arm 122b further includes a plurality of second splines 122f disposed at spaced-apart locations about a circumference of input arm 122b and which extend longitudinally along inner shaft 122. First and second pluralities of splines 122e, 122f are disposed adjacent to connecting arm 122c at an intermediate portion 122g of inner shaft 122 that is disposed between outer and inner coupling ends 122h, 122i of inner shaft 122. Outer and inner coupling ends 122h, 122i of inner shaft 122 may be circular and/or non-circular, but are configured to fixedly (e.g., non-rotatably) couple to respective shaft openings 112f, 114f of output and input couplers 112, 114, for instance, via interference-fit, welding, or any other suitable coupling technique. In this regard, inner shaft 122 is configured to rotate with output and input couplers 112, 114.

Outer shaft 124 of shaft assembly 120 includes an output end 124a and an input end 124b, and defines a central lumen 124c. Outer shaft 124 includes a plurality of first spline slots 124d at spaced-apart locations about the inner surface of outer shaft 124 at output end 124a of outer shaft 124 for receiving the plurality of first splines 122e of inner shaft 122 and torsionally locking output end 124a of outer shaft 124 to output arm 122a of inner shaft 122. Outer shaft 124 further includes a plurality of second spline slots 124e at spaced-apart locations about the inner surface of outer shaft 124 at input end 124b of outer shaft 124. The plurality of second spline slots 124e are larger than the plurality of first spline slots 124d such that each of the plurality of second spline slots 124e has a larger arc length about the circumference of central lumen 124c than each of the plurality of first spline slots 124d. Inner and outer shafts 122, 124 have non-round mating profiles. In particular, outer shaft 124 is configured to receive inner shaft 122 such that the plurality of first spline slots 124d receives the plurality of first splines 122e of shaft assembly 120 via interference-fit, and the plurality of second spline slots 124e slidably receives the plurality of second splines 122f via a close-fitting, slip-fit to enable partial angular deflection between inner and outer shafts 122, 124 for low torque output and partial locking engagement between inner and outer shafts 122, 124 for high torque output.

The plurality of second splines 122f of inner shaft 122 is configured rotate through the plurality of second spline slots 124e of outer shaft 124, as indicated by arrows “A”, so that inner shaft 122 rotates relative to outer shaft 124 until side walls 122g of the plurality of second splines 122f rotates into engagement with side walls 124g of the plurality of second spline slots 124e. Notably, such movement is bi-directional in that such rotation can occur in clockwise and/or counterclockwise directions through a given amount of play defined by the difference between an arc length of one of the second spline slots 124e of plurality of second spline slots 124e of outer shaft 124 and an arc length of one of the second splines 122f of the plurality of second splines 122f of inner shaft 122 (e.g., fatigue limiting deflection). Such relative rotational movement between inner and outer shafts 122, 124 provides a low torque flexure such that flexure deflection is within a low torque range. The range can be configured through design. As an example, low torque range can be set below 200 mNm. An amount of angular play is determined as per fatigue strength of the low torque flexure. Once sidewalls 122h of the plurality of second splines 122f engage sidewalls 124g of the plurality of second spline slots 124e of outer shaft 124 (on either side of second spline slots 124e, depending on direction of rotation), inner and outer shafts 122, 124 rotate together, as indicated by arrows “B” for providing high torque flexure such that flexure deflection is within a high torque range. Similar to the low torque range, the higher range can be set, for example, to equal to or greater than 200 mNm and less than or equal to 1000 mNm. Advantageously, low torque flexure can be more sensitive and precise without concerns of plastic deformation due to overloading or for fatigue life concerns. In particular, this dual concentric shaft assembly 120 provides low and high range torque measurements with higher fatigue limits. As inner shaft 122 and/or outer shaft 124 rotate, primary and/or secondary rotational angle sensors 116, 118 determine rotational angles of inner and/or outer shafts 122, 124 to determine an amount of rotation and/or torque applied to torque sensor 107, an amount of torque output from motors 106, and/or an amount of torque input to surgical instrument 60. In particular, primary and secondary rotational angle sensors 116, 118 measure torque by determining a difference of measured angular deflection between primary and secondary rotational angle sensors 116, 118.

In aspects, outer shaft 124 and/or inner shaft 122 may further define one or more pin holes 124f therethrough for receiving one or more lock pins (not shown) for coupling outer shaft 124 to inner shaft 122 and/or for providing a fatigue torque lock pin, which may be provided in addition to, or in place of, the plurality of second splines 122f and the plurality of second spline slots 124e. In aspects, inner shaft 122 and/or outer shaft 124 does not include any pin holes 124f. One or more pin holes defined through inner shaft 122 and/or outer shaft 124 proximate the second splines 122f, may be circular and/or elongated in a clockwise and/or counterclockwise direction (e.g., elliptical) about inner shaft 122 and/or outer shaft 124 (e.g., about the same arc length as one of the plurality of second spline slots 124e) for enabling relative rotation between inner and outer shafts 122, 124 and low torque flexure similar to that described above. Outer shaft 124, which may be in the form of a tube, enables a more precise level of sensor precision and sensitivity from inner shaft 122 by protecting it from high torsional load fatigue or plastic deformation overloading damage.

In aspects, lubrication may be provided between inner and outer shafts 122, 124, such as between splines 122f of inner shaft 122 and slots 124e of outer shaft 124, to reduce any frictional variation factors between inner and outer shafts 122, 124.

In aspects, primary and/or secondary rotational angle sensors 116, 118 may include any suitable high-resolution angle sensing structure such as an optical rotary encoder, a magnetic rotary encoder, etc.

In aspects, any of the disclosed splines and/or slots of torque sensor 107 can have any suitable mating forms including non-round shapes and/or configurations, regardless of concentricity, and in some aspects, may be concentric and mirrored in shape and/or configuration. These mating forms may be, but are not limited to, any single or combination of one or more flat surfaces, straight knurl, star forms, lobed forms, oval forms, clover forms, spur forms, helix forms, single forms, and/or multiple keyed forms.

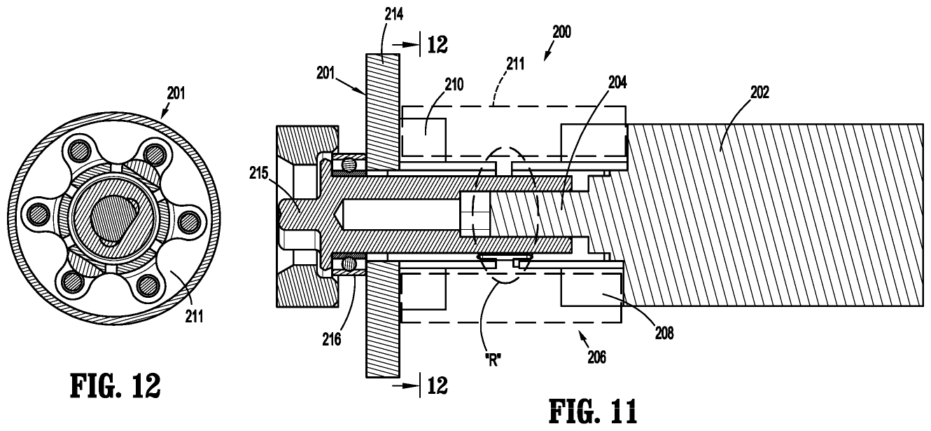

With reference to FIGS. 11 and 12, in aspects, a drive assembly 200 for robotic surgical system 10 includes a torque sensor assembly 201 coupled to a servo-drive motor 202. Motor 202 has a servo-drive output drive shaft 204. Torque sensor 201 includes a hollow reaction torque sensor 206. Hollow reaction torque sensor 206 includes a proximal reaction torque sensor mounting flange 208 and a distal reaction torque sensor mounting flange 210 that support a structural sleeve 211 and enable structural sleeve 211 to slide over proximal and distal reaction torque sensor mounting flanges 208, 210. Torque sensor 201 further includes a sensitive and vulnerable strain gage structural member region “R”. Structural sleeve 211 provides torsional overload protection for the sensitive and vulnerable strain gage structural members “R.” Torque sensor 201 further includes a motor plate distal mount 214. Torque sensor 201 further includes a device output drive coupler 215 that couples to servo-drive output drive shaft 204. Output drive coupler 215 is further supported by a ball bearing assembly 216.

The disclosed structure can include any suitable mechanical, electrical, and/or chemical components for operating the disclosed system or components thereof. For instance, such electrical components can include, for example, any suitable electrical and/or electromechanical, and/or electrochemical circuitry, which may include or be coupled to one or more printed circuit boards. As appreciated, the disclosed computing devices (and/or servers) can include, for example, a “controller,” “processor,” “digital processing device” and like terms, and which are used to indicate a microprocessor or central processing unit (CPU). The CPU is the electronic circuitry within a computer that carries out the instructions of a computer program by performing the basic arithmetic, logical, control and input/output (I/O) operations specified by the instructions, and by way of non-limiting examples, include server computers. In some aspects, the controller includes an operating system configured to perform executable instructions. The operating system is, for example, software, including programs and data, which manages hardware of the disclosed apparatus and provides services for execution of applications for use with the disclosed apparatus. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some aspects, the operating system is provided by cloud computing.

In some aspects, the term “controller” may be used to indicate a device that controls the transfer of data from a computer or computing device to a peripheral or separate device and vice versa, and/or a mechanical and/or electromechanical device (e.g., a lever, knob, etc.) that mechanically operates and/or actuates a peripheral or separate device.

In aspects, the controller includes a storage and/or memory device. The storage and/or memory device is one or more physical apparatus used to store data or programs on a temporary or permanent basis. In some aspects, the controller includes volatile memory and requires power to maintain stored information. In various aspects, the controller includes non-volatile memory and retains stored information when it is not powered. In some aspects, the non-volatile memory includes flash memory. In certain aspects, the non-volatile memory includes dynamic random-access memory (DRAM). In some aspects, the non-volatile memory includes ferroelectric random-access memory (FRAM). In various aspects, the non-volatile memory includes phase-change random access memory (PRAM). In certain aspects, the controller is a storage device including, by way of non-limiting examples, CD-ROMs, DVDs, flash memory devices, magnetic disk drives, magnetic tapes drives, optical disk drives, and cloud-computing-based storage. In various aspects, the storage and/or memory device is a combination of devices such as those disclosed herein.

In various aspects, the memory can be random access memory, read-only memory, magnetic disk memory, solid state memory, optical disc memory, and/or another type of memory. In various aspects, the memory can be separate from the controller and can communicate with the processor through communication buses of a circuit board and/or through communication cables such as serial ATA cables or other types of cables. The memory includes computer-readable instructions that are executable by the processor to operate the controller. In various aspects, the controller may include a wireless network interface to communicate with other computers or a server. In aspects, a storage device may be used for storing data. In various aspects, the processor may be, for example, without limitation, a digital signal processor, a microprocessor, an ASIC, a graphics processing unit (“GPU”), field-programmable gate array (“FPGA”), or a central processing unit (“CPU”).

The memory stores suitable instructions and/or applications, to be executed by the processor, for receiving the sensed data (e.g., sensed data from sensors 107). Although illustrated as part of the disclosed structure, it is also contemplated that a controller may be remote from the disclosed structure (e.g., on a remote server), and accessible by the disclosed structure via a wired or wireless connection. In aspects where the controller is remote, it is contemplated that the controller may be accessible by, and connected to, multiple structures and/or components of the disclosed system.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on the disclosed controllers or on a user device, including for example, on a mobile device, an IOT device, or a server system.

In some aspects, the controller includes a display to send visual information to a user. In various aspects, the display is a cathode ray tube (CRT). In various aspects, the display is a liquid crystal display (LCD). In certain aspects, the display is a thin film transistor liquid crystal display (TFT-LCD). In aspects, the display is an organic light emitting diode (OLED) display. In certain aspects, on OLED display is a passive-matrix OLED (PMOLED) or active-matrix OLED (AMOLED) display. In aspects, the display is a plasma display. In certain aspects, the display is a video projector. In various aspects, the display is interactive (e.g., having a touch screen) that can detect user interactions/gestures/responses and the like. In some aspects, the display is a combination of devices such as those disclosed herein.

The controller may include or be coupled to a server and/or a network. As used herein, the term “server” includes “computer server,” “central server,” “main server,” and like terms to indicate a computer or device on a network that manages the disclosed apparatus, components thereof, and/or resources thereof. As used herein, the term “network” can include any network technology including, for instance, a cellular data network, a wired network, a fiber-optic network, a satellite network, and/or an IEEE 802.11a/b/g/n/ac wireless network, among others.

In various aspects, the controller can be coupled to a mesh network. As used herein, a “mesh network” is a network topology in which each node relays data for the network. All mesh nodes cooperate in the distribution of data in the network. It can be applied to both wired and wireless networks. Wireless mesh networks can be considered a type of “Wireless ad hoc” network. Thus, wireless mesh networks are closely related to Mobile ad hoc networks (MANETs). Although MANETs are not restricted to a specific mesh network topology, Wireless ad hoc networks or MANETs can take any form of network topology. Mesh networks can relay messages using either a flooding technique or a routing technique. With routing, the message is propagated along a path by hopping from node to node until it reaches its destination. To ensure that all its paths are available, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. This concept can also apply to wired networks and to software interaction. A mesh network whose nodes are all connected to each other is a fully connected network.

In some aspects, the controller may include one or more modules. As used herein, the term “module” and like terms are used to indicate a self-contained hardware component of the central server, which in turn includes software modules. In software, a module is a part of a program. Programs are composed of one or more independently developed modules that are not combined until the program is linked. A single module can contain one or several routines, or sections of programs that perform a particular task.

As used herein, the controller includes software modules for managing various aspects and functions of the disclosed system or components thereof.

The disclosed structure may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

The phrases “in an aspect,” “in aspects,” “in various aspects,” “in some aspects,” “in other aspects” or the like may each refer to one or more of the same or different aspects in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).” Various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to all such states. Reference to a program may encompass the actual instructions and/or the intent of those instructions.

Securement of any of the components of the disclosed devices may be effectuated using known securement techniques such welding, crimping, gluing, fastening, etc.

Persons skilled in the art will understand that the structures and methods specifically described herein and shown in the accompanying figures are non-limiting exemplary aspects, and that the description, disclosure, and figures should be construed merely as exemplary of aspects. It is to be understood, therefore, that this disclosure is not limited to the precise aspects described, and that various other changes and modifications may be effectuated by one skilled in the art without departing from the scope or spirit of the disclosure. Additionally, the elements and features shown or described in connection with certain aspects may be combined with the elements and features of certain other aspects without departing from the scope of this disclosure, and that such modifications and variations are also included within the scope of this disclosure. Accordingly, the subject matter of this disclosure is not limited by what has been particularly shown and described.

1. A robotic surgical system, comprising:
an instrument drive assembly supporting a motor assembly;
a surgical instrument operably coupled to the instrument drive assembly; and
a torque sensor supported between the motor assembly and the surgical instrument, the torque sensor including:
an output coupler;
an input coupler; and
a shaft assembly that connects the output coupler to the input coupler, the shaft assembly including an outer shaft mounted on an inner shaft, the inner shaft movable relative to the outer shaft to impart a first torque, and the inner shaft movable with the outer shaft to impart a second torque.

2. The robotic surgical system of claim 1, wherein the first torque is lower than the second torque.

3. The robotic surgical system of claim 2, wherein the inner shaft includes an output arm and an input arm that are separated by a connecting arm.

4. The robotic surgical system of claim 3, wherein the connecting arm has a smaller diameter than the output arm and the input arm.

5. The robotic surgical system of claim 2, wherein the inner shaft includes a first spline supported on the output arm and a second spline supported on the input arm.

6. The robotic surgical system of claim 5, wherein the outer shaft defines a first spline slot within which the first spline seats and a second spline slot within which the second spline seats.

7. The robotic surgical system of claim 6, wherein the first spline and the first spline slot are torsionally locked together, and wherein the second spline is configured to slide through the second spline slot as the inner shaft rotates relative to the outer shaft.

8. The robotic surgical system of claim 7, wherein when the second spline slides through the second spline slot, the inner shaft is positioned for angular displacement relative to the outer shaft.

9. The robotic surgical system of claim 8, wherein the second spline is engageable with a sidewall of the outer shaft that defines the second spline slot, and wherein when the second spline engages the sidewall, the outer shaft and the inner shaft rotate together.

10. The robotic surgical system of claim 1, further comprising at least on rotational angle sensor coupled to the shaft assembly.

11. A surgical system, comprising:
an instrument drive assembly supporting a motor assembly; and
a torque sensor coupled to the motor assembly and including:
an output coupler configured to engage with a surgical instrument;
an input coupler engaged with the motor assembly; and
a shaft assembly that connects the output coupler to the input coupler, the shaft assembly including an outer shaft mounted on an inner shaft, the inner shaft movable relative to the outer shaft to impart a first torque, and the inner shaft movable with the outer shaft to impart a second torque.

12. The surgical system of claim 11, wherein the first torque is lower than the second torque.

13. The surgical system of claim 12, wherein the inner shaft includes an output arm and an input arm that are separated by a connecting arm.

14. The surgical system of claim 13, wherein the connecting arm has a smaller diameter than the output arm and the input arm.

15. The surgical system of claim 12, wherein the inner shaft includes a first spline supported on the output arm and a second spline supported on the input arm.

16. The surgical system of claim 15, wherein the outer shaft defines a first spline slot within which the first spline seats and a second spline slot within which the second spline seats.

17. The surgical system of claim 16, wherein the first spline and the first spline slot are torsionally locked together, and wherein the second spline is configured to slide through the second spline slot as the inner shaft rotates relative to the outer shaft.

18. The surgical system of claim 17, wherein when the second spline slides through the second spline slot, the inner shaft is positioned for angular displacement relative to the outer shaft.

19. The surgical system of claim 18, wherein the second spline is engageable with a sidewall of the outer shaft that defines the second spline slot, and wherein when the second spline engages the sidewall, the outer shaft and the inner shaft rotate together.

20. A torque sensor for a robotic surgical system, the torque sensor comprising:
an output coupler configured to engage with a surgical instrument;
a first rotational angle sensor coupled to the output coupler;
an input coupler configured to engage with a motor assembly;
a second rotational angle sensor coupled to the input coupler; and
a shaft assembly defining a longitudinal axis and connecting the output coupler to the input coupler, the shaft assembly including an outer shaft mounted on an inner shaft, the inner shaft movable relative to the outer shaft through a first angular displacement, the inner shaft movable with the outer shaft through a second angular displacement that is greater than the first angular displacement, wherein the first and second rotational angle sensors are configured to cooperate with one another to enable the robotic surgical system to determine torque imparted on the robotic surgical system as the shaft assembly rotates about the longitudinal axis.
